(19) United States
(12) Patent Application Publication
(10) Pub. No.: US 2024/0023639 A1
(43) Pub. Date: Jan. 25, 2024

(54) COMPRESSION SOCK AND FOOT MEASURING SYSTEM HAVING THE SAME

(71) Applicant: NESI TRADING CO., LTD., Taichung City (TW)

(72) Inventor: Tsung-E LIAO, Taichung City (TW)

(21) Appl. No.: 17/813,967

(22) Filed: Jul. 21, 2022

(51) Int. Cl. A41B 11/02; A61B 5/107

(52) U.S. Cl. CPC A41B 11/02; A61B 5/1074

(57) ABSTRACT

A compression sock used in a foot measuring system, and worn on a user's foot that has a metatarsophalangeal joint area. The compression sock includes a sock cuff end, and a sock body. The sock cuff end is for insertion by the user's foot. The sock body is connected to the sock cuff end, and includes a high compression zone that surrounds the metatarsophalangeal joint area, and a standard compression zone that is proximate to the sock cuff end. A compression pressure value exerted by the high compression zone of the sock body on the metatarsophalangeal joint area is higher than a compression pressure value exerted by the standard compression zone on the rest of the user's foot.

### FIELD

The disclosure relates to a compression sock, and more particularly to a foot measuring system using the compression sock.

### BACKGROUND

When making shoe purchases, consumers generally follow one of several shoe-size systems to determine their shoe-size. However, the size of shoes made by different shoe manufacturers may be different for the same shoe-size. Consequently, consumers may have difficulty finding shoes that fit correctly, when they purchase shoes according to their own shoe-size on the conventional shoe-size systems.

FIG. 1 shows a foot scanning system 1 disclosed in US Patent Application Publication No. US20170169571A1. The foot scanning system includes a detection area 11 adapted for placement of a user's foot 10, a reference device 12 used to secure the user's foot 10, a depth camera 13 used to capture an image of the user's foot 10 that is placed on the detection area 11, and a processing device 14 coupled with the depth camera 13. The processing device 14 processes the image captured by the depth camera 13 to calculate parameters of the user's foot 10 and obtain a 3D model based on the relative position between the reference device 12 and the camera 13.

As shown in FIG. 1, the user is in a standing position and the user's foot 10 is barefoot when used with the foot scanning system 1. However, in the standing position, posture and the center of gravity of the user may affect the accuracy of the results, and cause deviation between each measurement which makes it difficult to determine a correct shoe-size for the user's foot 10. Additionally, because the user's foot 10 is barefoot, variables such as splaying of the toes and peripheral edema may affect the accuracy of the results, and may become a hygiene concern for other users of the foot scanning system 1.

### SUMMARY

Therefore, an object of the disclosure is to provide a compression sock and a foot measuring system that can alleviate at least one of the drawbacks of the prior art.

Accordingly, one aspect of the disclosure is to provide a compression sock which is adapted for use in a foot measuring system and which is adapted to be worn on a user's foot that has a metatarsophalangeal joint area, a toe area, a lateral ball point, and a medial ball point. The compression sock includes a sock cuff end, a sock body, and a sock toe end. The sock cuff end is adapted for insertion by the user's foot. The sock body is connected to the sock cuff end, and includes a high compression zone that surrounds the metatarsophalangeal joint area of the user's foot, and a standard compression zone that is proximate to the sock cuff end, and that connects with the high compression zone. A compression pressure value exerted by the high compression zone of the sock body on the metatarsophalangeal joint area of the user's foot is higher than a compression pressure value exerted by the standard compression zone on the rest of the user's foot. The sock toe end is connected with the sock body opposite to the sock cuff end for receiving the toe area of the user's foot.

Another aspect of the disclosure is to provide a foot measuring system which is adapted for measuring the user's foot, and which includes the compression sock, and a measuring unit. The measuring unit performs measurements of the high compression zone of the sock body worn on the user's foot for obtaining one of a ball girth value and a ball width value of the user's foot.

### BRIEF DESCRIPTION OF THE DRAWINGS

Other features and advantages of the disclosure will become apparent in the following detailed description of the embodiments with reference to the accompanying drawings, of which:

FIG. 1 is a schematic and perspective view illustrating a foot scanning system disclosed in US Patent Application Publication No. US20170169571A1;

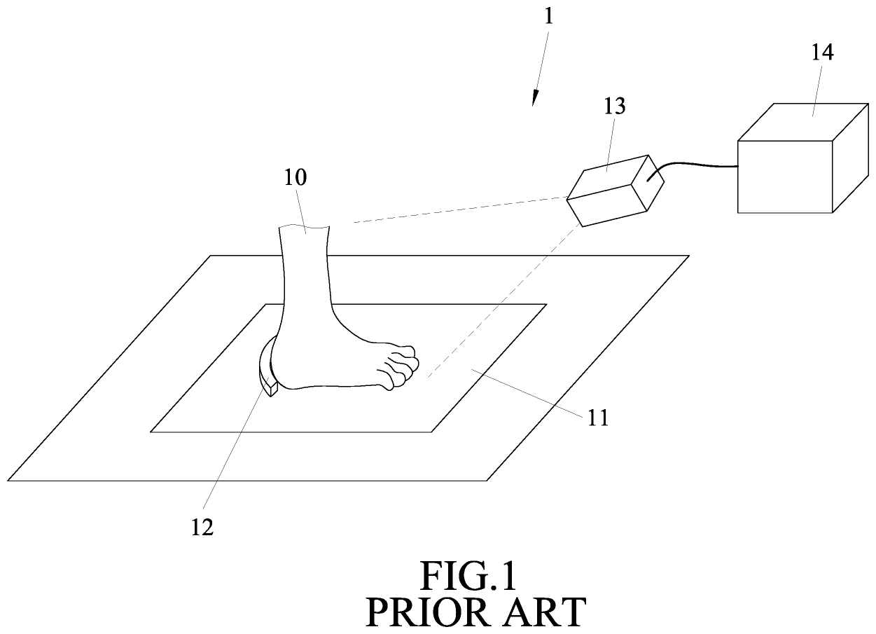

FIG. 2 is a schematic and perspective view illustrating an embodiment of a compression sock and a foot measuring system according to the disclosure;

FIG. 3 is a top view illustrating the embodiments;

FIG. 4 is a perspective view illustrating the embodiment;

FIG. 5 is a top view showing the positions of a high compression zone and a standard compression zone of the embodiment relative to corresponding bone parts of a foot; and

FIG. 6 is a top view of a variation of the embodiment of the compression sock.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

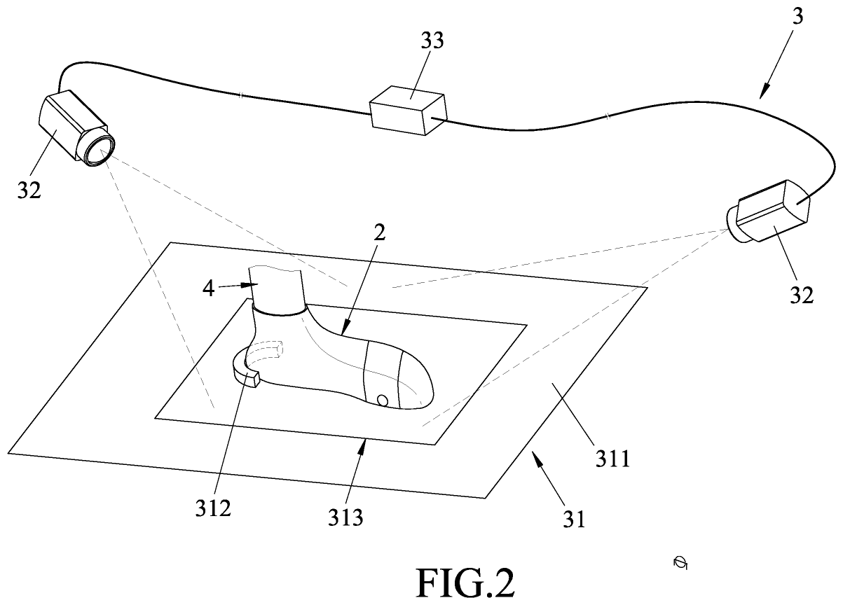

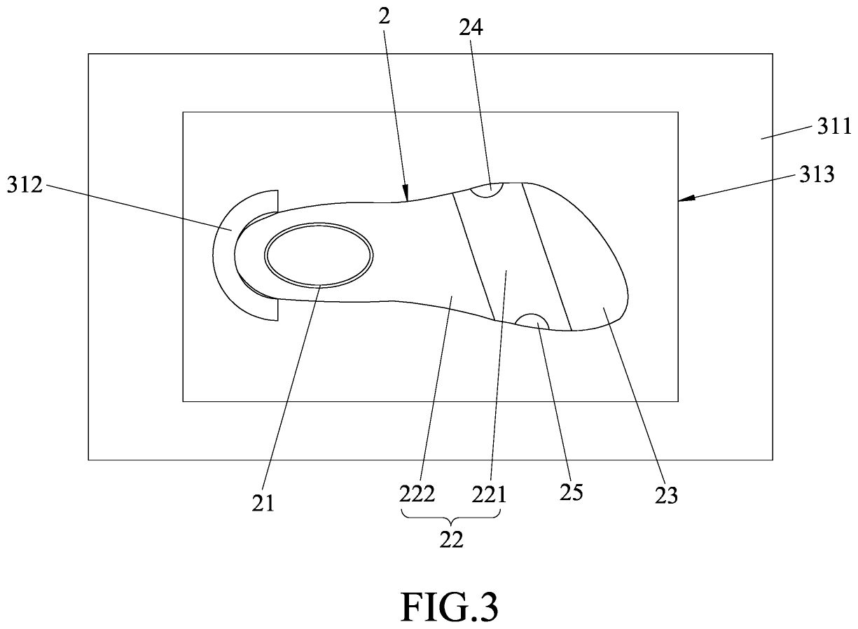

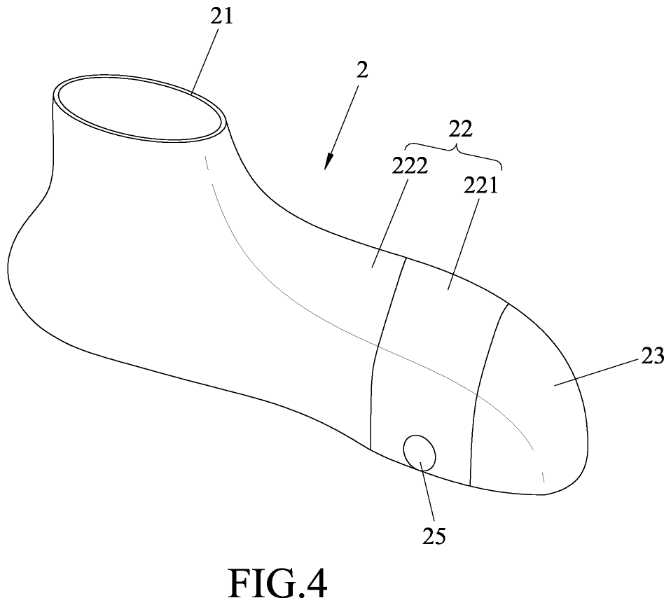

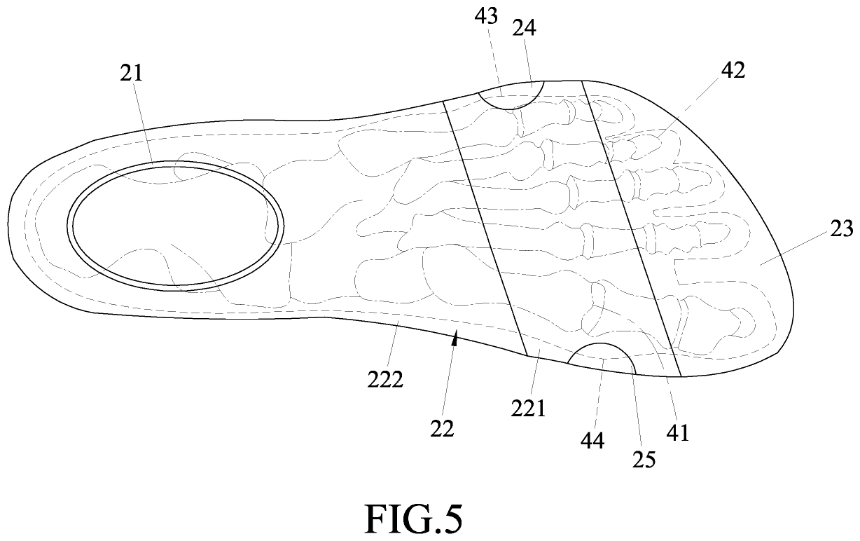

Referring to FIGS. 2 to 5, an embodiment of a compression sock 2 is shown. The compression sock 2 is adapted for use in a foot measuring system 3, and is adapted to be worn on a user's foot 4. The compression sock 2 includes a sock cuff end 21, a sock body 22, a sock toe end 23, a first tag 24, and a second tag 25. The foot measuring system 3 is adapted for measuring the user's foot 4, and includes a measuring unit 31, at least one camera 32, and a processing device 33. The user's foot 4 has a metatarsophalangeal joint area 41, a toe area 42, a lateral ball point 43, and a medial ball point 44.

The compression sock 2 is made of one of a natural fiber material, a synthetic fiber material, and a composite fiber material. In some embodiments, the compression sock 2 is made of a latex polymer material and manufactured through a one piece molding process.

The sock cuff end 21 is adapted for insertion by the user's foot 4 and wraps around and secures an ankle of the user's foot 4. In some embodiments, the sock cuff end 21 can wrap around a sole of the user's foot 4 and be secured around a mid point of a sole of the user's foot 4.

The sock body 22 is connected to the sock cuff end 21, and includes a high compression zone 221 that surrounds the metatarsophalangeal joint area 41 of the user's foot 4, and a standard compression zone 222 that is proximate to the sock cuff end 21, and that connects with the high compression zone 221. A compression pressure value exerted by the high compression zone 221 of the sock body 22 on the metatarsophalangeal joint area 41 of the user's foot 4 is higher than a compression pressure value exerted by the standard compression zone 222 on the rest of the user's foot 4. In this embodiment, the compression pressure value exerted by the high compression zone 221 on the metatarsophalangeal joint area 41 of the user's foot 4 is 28±3 mmHg, and the compression pressure value exerted by the standard compression zone 222 on the rest of the user's foot 4 is 15±3 mmHg. It should be noted, that the sock cuff end 21 and the sock toe end 23 also each exert a compression pressure value of 15±3 mmHg on their corresponding areas of the user's foot 4.

The sock toe end 23 is connected with the sock body 22 opposite to the sock cuff end 21 for receiving the toe area 42 of the user's foot 4.

The first tag 24 is disposed on an outer surface of the sock body 22 corresponding in position to the lateral ball point 43 of the user's foot 4. The second tag 25 is disposed on an outer surface of the sock body 22 corresponding in position to the medial ball point 44 of the user's foot 4. Each of the first tag 24 and the second tag 25 is disposed on the sock body 22 via one of a stitching method, a dying method, and an attaching method using a releasable fastener such as a hook-and-loop fastener or a sticker. Thus, each of the first tag 24 and the second tag 25 can be one of a stitched part, a dyed part, and a releasably attached part. However, this is not a limitation and other methods of attaching the first tag 24 and the second tag 25 to the sock body 22 may be used. In this embodiment, the first tag 24 and the second tag 25 are circular, and are disposed on the sock body 22 via hook-and-loop fasteners or stickers after the compression sock 2 has already been put on the user's foot 4, so that variations in each user's foot 4 may be taken into account and the first and second tags 24, 25 may be accurately placed on the compression sock 2 to respectively correspond to the lateral ball point 43 and the medial ball point 44.

Additionally, it should be mentioned that while the first tag 24 and the second tag 25 in this embodiment are releasably attached parts for identifying the position of the lateral ball point 43 and the medial ball point 44, they can be dyed parts in other embodiments. In an example, the dyed parts may contain a piezochromic material (not shown), by which the dyed parts may be used to indicate the position of the lateral ball point 43 and the medial ball point 44. Since the lateral ball point 43 and the medial ball point 44 are protrusions of the user's foot 4 the compression sock 2 employing the piezochromic material will change color due to the changes in pressure caused by the lateral ball point 43 and the medial ball point 44 when the compression sock 2 is put on the user's foot 4. Consequently, the position of the lateral ball point 43 and the medial ball point 44 of the user's foot 4 may be identified by the color change.

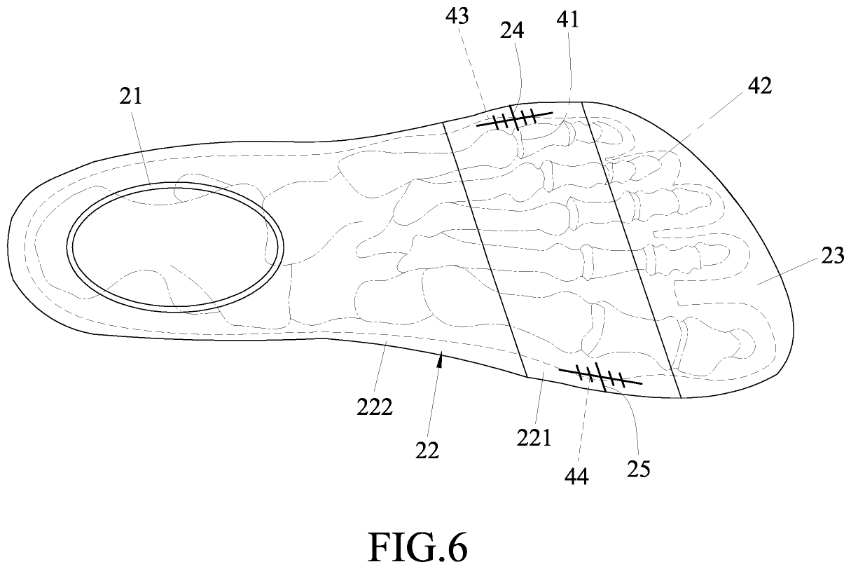

Referring to FIG. 6, showing a variation of the embodiment, each the first tag 24 and the second tag 25 has a striped pattern and is disposed on the high compression zone 221 of the sock body 22 via one of a sewing method and a dying method. The striped patterns of the first tag 24 and the second tag 25 are used for ease of identification. The foot measuring system 3 is able to identify locations and positions of the lateral ball point 43 and the medial ball point 44 based on changes in shape of the striped pattern, when the toe area 42 of the user's foot 4 flexes.

The measuring unit 31 includes a base plate 311, and a positioning member 312. The base plate 311 has a detection area 313 for the user's foot 4 wearing the compression sock 2 to be placed. The positioning member is disposed on the detection area 313 to secure the ankle of the user's foot 4. In this embodiment, the positioning member 312 has a U-shaped structure to ergonomically fit with the ankle of the user's foot 4. However, this is not a limitation of the disclosure and the positioning member 312 can be in different structures to ergonomically fit with other areas of the user's foot 4, such as the toe area 42 or the metatarsophalangeal joint area 41.

The at least one camera 32 may be a depth camera or a 3D camera that has a photosensor such as a CCD (charge coupled device) or a CMOS (complementary metal oxide semiconductor transistor) and that can take images from an object and capture image data including depth data or other three dimensional data. In this embodiment, the at least one camera 32 is for capturing image data of the compression sock 2 when the compression sock 2 is worn on the user's foot 4. The image data is then sent to a processing device 33 which is coupled with the at least one camera 32 and which analyzes the image data. In this embodiment, there are two cameras 32 and they are both depth cameras, however, in other embodiments, there may be more depth cameras and the number of the depth cameras is not limited to the disclosed amount. The depth cameras 32 are used to shoot to the detection area 313 to capture image data of the foot 4 wearing the compression sock 2 and the positioning member 312, and send the same to the processing device 33. It should be noted, that the compression sock 2 worn on the user's foot 4 can prevent infrared radiation from penetrating the skin surface of the user's foot 4 and causing the captured image to be inaccurate.

The processing device 33 is coupled with the depth cameras 32 and analyzes the image data to obtain various parameters relating to the user's foot 4, such as a ball width value, a ball girth value, a waist girth value, and an instep girth value etc. However, the disclosure is not limited to these parameters and other parameters may be obtained from the image data captured by the cameras 32. For obtaining the ball width value and the ball girth value of the user's foot 4, the measuring unit 31 performs measurements of the high compression zone 221 of the sock body 22 worn on the user's foot 4. Specifically, the camera 32 captures image data of the high compression zone 221 including the first and second tags 24, 25. The processing device 33 then analyzes the image data, identifies locations and positions of the first and second tags 24, 25, and calculates the ball girth value and the ball width value of the user's foot 4 based on the locations and positions of the first and second tags 24, 25, wherein the locations and positions of the first and second tags 24, 25 respectively represent locations and positions of the lateral ball point 43 and the medial ball point 44 of the user's foot 4 and are used as parameters for calculating the ball girth value and the ball width value.

Here, the ball width value is defined as the straight-line distance between the lateral ball point 43 and the medial ball point 44, and the ball girth value is the circumference extending around the lateral ball point 43 and the medial ball point 44 of the user's foot 4. By having the high compression zone 221 of the compression sock 2 exerting a higher compression pressure value on the metatarsophalangeal joint area 41 of the user's foot 4 to tighten the high compression zone 221 to the metatarsophalangeal joint area 41 and to more clearly delineate the outline of the user's foot 4, and having the first and second tags 24, 25 to clearly label the positions of the lateral ball point 43 and the medial ball point 44, the processing unit 33 can accurately identify the locations and positions of the lateral ball point 43 and the medial ball point 44 during its analysis and calculation for the ball girth value and the ball width value of the user's foot 4.

It should be noted, that the compression sock 2 is not limited to being used with the foot measuring system 3 of the disclosure, and can be adapted for use in other foot shape measurement systems, foot shape measuring devices, and foot shape measuring methods, such as those disclosed in CN203220012U (FOOT CIRCUMFERENCE MEASURING SCALE), USD840253S1 (FOLDABLE FOOT MEASURING DEVICE), WO2018065803A1 (FOOT SHAPE ACQUISITION USING DEPTH SENSOR AND PRESSURE PLATE TECHNOLOGY), WO2020208298A1 (AN APPARATUS, A SYSTEM AND A METHOD OF FOOT MEASUREMENT FOR SELECTING READY-MADE FOOTWEAR), U.S. Pat. No. 2,942,344A (FOOT MEASURING DEVICE), GB2509696A (FOOT MEASURING SYSTEM), U.S. Pat. No. 6,834,437B1 (FOOT MEASUREMENT SYSTEM), and US20040143452A1 (SYSTEM AND METHOD OF DYNAMICALLY ASSESSING FOOT CHARACTERISTICS). However, applications of the compression sock 2 is not limited to the above.

In summary of the above, in the compression sock 2 of the present disclosure, by virtue of the high compression zone 221 exerting a higher compression pressure value on the metatarsophalangeal joint area 41 of the user's foot, and the standard compression zone also exerting a compression pressure value on the rest of the user's foot 4, the user's foot 4 is delineated more clearly, and accurate and consistent repeat measurements can be made by the foot measuring system 3 so that appropriate footwear may be chosen to fit the user's foot 4. Additionally, hygienic conditions may be maintained when using the foot measuring system 3 with the compression sock 2, and infrared radiation from the camera 32 can be prevented from penetrating the user's foot 4 and causing the captured depth image to be inaccurate.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A compression sock adapted for use in a foot measuring system, and adapted to be worn on a user's foot that has a metatarsophalangeal joint area, a toe area, a lateral ball point, and a medial ball point, said compression sock comprising:
a sock cuff end adapted for insertion by the user's foot;
a sock body connected to said sock cuff end, and including a high compression zone that surrounds the metatarsophalangeal joint area of the user's foot, and a standard compression zone that is proximate to said sock cuff end, and that connects with said high compression zone, wherein a compression pressure value exerted by said high compression zone of said sock body on the metatarsophalangeal joint area of the user's foot is higher than a compression pressure value exerted by said standard compression zone on the rest of the user's foot; and
a sock toe end connected with said sock body opposite to said sock cuff end for receiving the toe area of the user's foot.

2. The compression sock as claimed in claim 1, wherein said compression pressure value exerted by said high compression zone on the metatarsophalangeal joint area of the user's foot is 28±3 mmHg, and said compression pressure value exerted by said standard compression zone on the rest of the user's foot is 15±3 mmHg.

3. The compression sock as claimed in claim 1 further comprising a first tag disposed on an outer surface of said sock body corresponding in position to the lateral ball point of the user's foot, and a second tag disposed on an outer surface of said sock body corresponding in position to the medial ball point of the user's foot.

4. The compression sock as claimed in claim 3, wherein said first tag and said second tag are circular.

5. The compression sock as claimed in claim 3, wherein each of said first tag and said second tag has a striped pattern.

6. The compression sock as claimed in claim 1, wherein each of said first tag and said second tag is disposed on said sock body via one of a stitching method, a dying method, and an attaching method using a releasable fastener.

7. The compression sock as claimed in claim 3, wherein each of said first tag and said second tag is one of a stitched part, a dyed part, and a releasably attached part.

8. The compression sock as claimed in claim 7, wherein each of said first tag and said second tag is a dyed part that contains a piezochromic material.

9. The compression sock as claimed in claim 1, wherein said compression sock is made of a latex polymer material.

10. The compression sock as claimed in claim 1, wherein said compression sock is made of one of a natural fiber material, a synthetic fiber material, and a composite fiber material.

11. A foot measuring system adapted for measuring a user's foot, said foot shape measuring system comprising:
said compression sock as claimed in claim 1;
a measuring unit which performs measurements of said high compression zone of said sock body worn on the user's foot for obtaining one of a ball girth value and a ball width value of the user's foot.

12. The foot measuring system as claimed in claim 11, wherein:
said compression sock further includes a first tag disposed on an outer surface of said sock body corresponding in position to the lateral ball point of the user's foot, and a second tag disposed on an outer surface of said sock body corresponding in position to the medial ball point of the user's foot; and
said measuring unit includes
at least one camera for capturing image data of said high compression zone of said compression sock worn on the user's foot, and
a processing device which is coupled with said at least one camera and which analyzes said image data, identifies locations and positions of said first and second tags, and calculates the ball girth value of the user's foot based on the locations and positions of said first and second tags, wherein the locations and positions of said first and second tags respectively represent locations and positions of the lateral ball point and the medial ball point of the user's foot and are used as parameters for calculating the ball girth value.

13. The foot measuring system as claimed in claim 11, wherein:
said compression sock further includes a first tag disposed on an outer surface of said sock body corresponding in position to the lateral ball point of the user's foot, and a second tag disposed on an outer surface of said sock body corresponding in position to the medial ball point of the user's foot; and
said measuring unit includes
at least one camera for capturing image data of said high compression zone of said compression sock worn on the user's foot, and
a processing device which is coupled with said at least one camera, and which analyzes said image data, identifies locations and positions of said first and second tags, and calculates the ball width value of the user's foot based on the locations and positions of said first and second tags, wherein the locations and positions of said first and second tags respectively represent locations and positions of the lateral ball point and the medial ball point of the user's foot and are used as parameters for calculating the ball width value.
